(19) United States
(12) Patent Application Publication
(10) Pub. No.: US 2022/0358652 A1
(43) Pub. Date: Nov. 10, 2022

(54) IMAGE PROCESSING APPARATUS, RADIATION IMAGING APPARATUS, IMAGE PROCESSING METHOD, AND STORAGE MEDIUM

(71) Applicant: CANON KABUSHIKI KAISHA, Tokyo (JP)

(72) Inventor: Sota TORII, Tokyo (JP)

(21) Appl. No.: 17/866,851

(22) Filed: Jul. 18, 2022

Related U.S. Application Data
(63) Continuation of application No. PCT/JP2021/002775, filed on Jan. 27, 2021.

(30) Foreign Application Priority Data
Jan. 29, 2020 (JP) 2020-012886
Jan. 26, 2021 (JP) 2021-010628

(51) Int. Cl. G06T 7/00; A61B 6/00

(52) U.S. Cl. CPC G06T 7/0012; A61B 6/482; A61B 6/56; G06T 2207/10116; G06T 2207/30008; G06T 2207/20081

(57) ABSTRACT

An image processing apparatus for processing a radiation image, comprises a calculation unit configured to calculate, in a calculation region, a physical amount representing a characteristic of a material, the calculation region being obtained using (a) a specific region regarding a specific material in an image representing the characteristic of the material and (b) a relative positional relationship of a radiation tube, a radiation detector, and an object, wherein the image representing the characteristic of the material is obtained using information about a plurality of radiation energies.

### CROSS-REFERENCE TO RELATED APPLICATIONS

This application is a Continuation of International Patent Application No. PCT/JP2021/002775, filed Jan. 27, 2021, which claims the benefit of Japanese Patent Application No. 2020-012886, filed Jan. 29, 2020, and Japanese Patent Application No. 2021-010628, filed Jan. 26, 2021, all of which are hereby incorporated by reference herein in their entirety.

### BACKGROUND OF THE INVENTION

### Field of the Invention

The present invention relates to an image processing apparatus, a radiation imaging apparatus, an image processing method, and a storage medium.

### Background Art

As an imaging apparatus used for medical image diagnosis by radiation, a radiation imaging apparatus using a flat panel detector (to be abbreviated as “FPD” hereinafter) has been widespread, and various applications have been developed and used practically.

To prevent a bone fracture, it is important to quantitatively measure the calcium amount in a bone, and this is known to contribute to early detection of osteoporosis. As a simple quantitative bone mineral measuring method with high measurement accuracy, DXA (Dual-energy X-ray Absorptiometry) has received a great deal of attention. In DXA, using two X-ray beams having different energy distributions detected by an FPD, a bone density can be measured based on the difference of an X-ray absorption coefficient between a soft tissue and a bone tissue. To measure the bone density, it is necessary to capture a small change over time. If an operator decides a region to measure the bone density, the bone density cannot correctly be measured because of the influence of variations between operators.

Japanese Patent Laid-Open No. 9-24039 (PATENT LITERATURE: PTL 1) discloses that a region of interest (ROI) to be calculated is automatically decided by histogram analysis of image signals, thereby suppressing variations in measurement caused by a human factor.

In PTL 1, it is described that a pencil beam or a fan beam is used as irradiation X-rays. If a fan beam is used, enlargement imaging is performed, in which an obtained image becomes larger than an actual object. Hence, the present inventor found that in a region of interest of an image obtained by enlargement imaging, it may be impossible to correctly obtain, using the technique of PTL 1, a physical amount (measurement value) representing the characteristic of a material, for example, a bone mineral amount. This also poses the same problem when not a fan beam but radiation like a cone beam with a spread is used.

In consideration of the above-described conventional technique, the present invention provides an image processing technique capable of more correctly calculating a physical amount representing the characteristic of a material forming an object even in enlargement imaging using a fan beam, a cone beam, or the like.

### SUMMARY OF THE INVENTION

According to one aspect of the present invention, there is provided an image processing apparatus for processing a radiation image, comprising a calculation unit configured to calculate, in a calculation region, a physical amount representing a characteristic of a material, the calculation region being obtained using (a) a specific region regarding a specific material in an image representing the characteristic of the material and (b) a relative positional relationship of a radiation tube, a radiation detector, and an object, wherein the image representing the characteristic of the material is obtained using information about a plurality of radiation energies.

According to another aspect of the present invention, there is provided an image processing apparatus for processing a radiation image, comprising a calculation unit configured to calculate, in a calculation region obtained using a range having a pixel value lower than a threshold in a specific region concerning a specific material in an image representing a characteristic of a material, which is obtained using information about a plurality of radiation energies, a physical amount representing the characteristic of the material.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a view showing an example of the configuration of a radiation imaging system according to the first embodiment;

FIG. 2A is a flowchart showing a processing procedure by an image processing unit according to the first embodiment;

FIG. 2B is a flowchart showing a modification of the processing procedure by the image processing unit according to the first embodiment;

FIG. 3 is a view showing a high-energy image, a low-energy image, a bone image, and a fat image, in which 3a is a view showing a high-energy radiation image, 3b is a view showing a low-energy radiation image, 3c is a view showing the material decomposition image of soft tissues, and 3d is a view showing the material decomposition image of bones;

FIG. 4 is a view for explaining the relative geometric arrangement of a radiation tube, an object, and an FPD;

FIG. 5 is a view showing an X-ray image obtained by capturing a lumbar spine phantom;

FIG. 6 is a view showing an effect according to the first embodiment;

FIG. 7 is a view showing an effect according to the first embodiment; and

FIG. 8 is a view for explaining a processing method according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

In the following embodiments and claims, radiation includes not only X-rays but also α-rays, β-rays, γ-rays, and various kinds of particle beams.

### First Embodiment

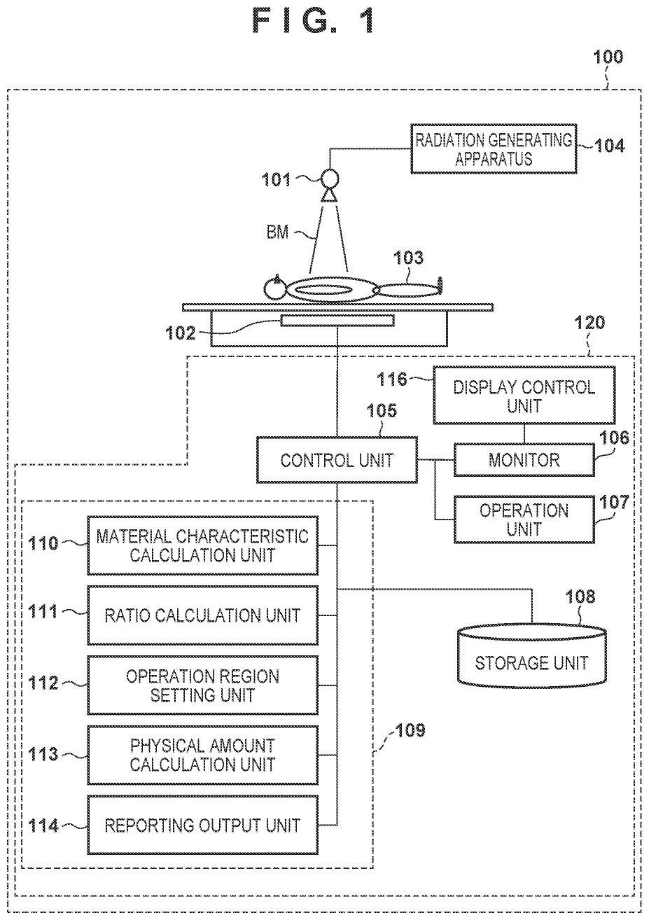

FIG. 1 is a block diagram showing an example of the configuration of a radiation imaging system 100 according to the first embodiment. The radiation imaging system 100 includes a radiation generating apparatus 104, a radiation tube 101, an FPD 102 (radiation detector), and an information processing apparatus 120. The information processing apparatus 120 processes information based on a radiation image obtained by capturing an object. Note that the configuration of the radiation imaging system 100 will be also simply referred to as a radiation imaging apparatus.

The radiation generating apparatus 104 applies a high-voltage pulse to the radiation tube 101 in accordance with a user operation on an exposure switch (not shown), thereby generating radiation. In the first embodiment, the type of radiation is not particularly limited. In medical image diagnosis, X-rays are mainly used. X-rays generated by the radiation generating apparatus 104 have a spread from the radiation tube 101 toward an object 103, like a fan beam or a cone beam (BM in FIG. 1), and some components of the radiation pass through the object 103 and reach the FPD 102.

An object included in an image obtained by the FPD 102 is captured larger than the actual object 103 by enlargement imaging. The FPD 102 includes a radiation detector including a pixel array configured to generate an image signal according to radiation. The FPD 102 accumulates charges based on the image signal to obtain a radiation image and transfers it to the information processing apparatus 120. In the radiation detector of the FPD 102, pixels each configured to output a signal according to incident light are arranged in an array (two-dimensional area). The photoelectric conversion element of each pixel converts radiation converted into visible light by a phosphor into an electrical signal, and outputs it as an image signal. The radiation detector of the FPD 102 is thus configured to detect radiation transmitted through the object 103 and obtain an image signal (radiation image).

The drive unit (not shown) of the FPD 102 outputs, to the control unit 105, an image signal (radiation image) read in accordance with an instruction from the control unit 105.

The information processing apparatus 120 processes information based on the radiation image obtained by capturing the object. The information processing apparatus 120 includes the control unit 105, a monitor 106, an operation unit 107, a storage unit 108, an image processing unit 109, and a display control unit 116.

The control unit 105 includes one or a plurality of processors (not shown), and executes programs stored in the storage unit 108, thereby implementing various kinds of control of the information processing apparatus 120. The storage unit 108 stores results of image processing and various kinds of programs. The storage unit 108 is formed by, for example, a ROM (Read Only Memory), a RAM (Random Access Memory), and the like. The storage unit 108 can store an image output from the control unit 105, an image processed by the image processing unit 109, and a calculation result in the image processing unit 109.

The image processing unit 109 processes the radiation image detected by the FPD 102. The image processing unit 109 includes, as functional components, a material characteristic calculation unit 110, a ratio calculation unit 111, an operation region setting unit 112, a physical amount calculation unit 113, and a reporting output unit 114 (output processing unit). These functional components may be implemented by the processor of the control unit 105 executing a predetermined program, or may be implemented using programs that one or a plurality of processors provided in the image processing unit 109 read out from the storage unit 108. Each of the processors in the control unit 105 and the image processing unit 109 is formed by, for example, a CPU (central processing unit). The configuration of each unit of the image processing unit 109 may be formed by an integrated circuit or the like if it can obtain the same function. The information processing apparatus 120 can also be configured to include, as its internal configuration, a graphic control unit such as a GPU (Graphics Processing Unit), a communication unit such as a network card, and an input/output control unit such as a keyboard, a display, or a touch panel.

The monitor 106 (display unit) displays the radiation image (digital image) that the control unit 105 received from the FPD 102 or an image processed by the image processing unit 109. The display control unit 116 controls the display of the monitor 106 (display unit). The operation unit 107 can input an instruction to the image processing unit 109 or the FPD 102, and accepts input of an instruction to the FPD 102 via a user interface (not shown).

In the above-described configuration, the radiation generating apparatus 104 applies a high voltage to the radiation tube 101 and irradiates the object 103 with radiation. The FPD 102 functions as an obtaining unit configured obtain a plurality of radiation images that are obtained by irradiating the object 103 with radiation and correspond to a plurality of energies. The FPD 102 generates two radiation images of different radiation energies by the radiation irradiation. The radiation images corresponding to the plurality of energies include a low-energy radiation image and a high-energy radiation image generated based on radiation energy higher than the low-energy radiation image.

Based on the plurality of radiation images obtained by the FPD 102, the material characteristic calculation unit 110 generates a material characteristic image capable of extracting the inside of the object 103 into the region of each material. The material characteristic calculation unit 110 functions as a specifying unit configured to specify a specific region made of a specific material in an image representing the characteristic of a material, which is generated based on radiation images of a plurality of energies obtained by radiation irradiation from the radiation tube to the object 103. The material characteristic calculation unit 110 can generates, as a material characteristic image, a material decomposition image or a material identification image. Here, a material decomposition image is an image obtained by, when the object 103 is expressed by two or more specific materials, decomposing the object into the two or more materials, each of which is formed by the thickness or density of the material. Also, a material identification image is an image obtained by, when the object 103 is expressed by one specific material, decomposing the object into the effective atomic number and the surface density of the material.

The ratio calculation unit 111 calculates the ratio of a specific region in an image (material characteristic image) representing the characteristic of a material based on a geometric arrangement representing the relative positional relationship of the radiation tube 101, the FPD 102 (radiation detector), and the object 103. If the image representing the characteristic of a material is, for example, a material decomposition image, the specific region is a material (a bone region or a fat region) forming the object 103.

The operation region setting unit 112 specifies a region made of one material decomposed from the radiation images corresponding to the plurality of energies obtained by radiation irradiation to the object 103. For example, the operation region setting unit 112 can calculate a bone region as a specific region from a bone image that is a material decomposition image.

The operation region setting unit 112 can use various methods as a region extraction method, and can use at least one of region extraction methods such as binarization, region extension, edge detection, graph cut, and paint. Also, machine learning using a lot of radiation images of the object 103 as supervisory data may be performed. The operation region setting unit 112 can specify a region made of one material using a region extraction method by machine learning for the plurality of radiation images obtained by the FPD 102. If radiation images of two energies are obtained, as in this embodiment, the above-described series of region extraction processes can accurately be executed by creating a bone image decomposing bones in advance.

In addition, the operation region setting unit 112 calculates, as an exclusion target region, a region where bones are captured thin by incidence (oblique incidence) of X-rays from an oblique direction. The operation region setting unit 112 excludes the calculated exclusion target region where bones are captured thin from the specific region (for example, a bone region), and sets the reduced specific region as a calculation region (region of interest) in the image (material decomposition image) representing the characteristic of the material.

The operation region setting unit 112 calculates, as the exclusion target region, a range having pixel values lower than a predetermined threshold in the specific region. Based on the calculated range (exclusion target region), the operation region setting unit 112 sets a calculation region to calculate a physical amount representing the characteristic of the material to the image representing the characteristic of the material. The operation region setting unit 112 excludes the calculated range (exclusion target region) from the specific region (for example, a bone region), and sets the reduced specific region as the calculation region in the image representing the characteristic of the material.

The operation region setting unit 112 can also set the calculation region to calculate the physical amount representing the characteristic of the material in the image representing the characteristic of the material based on the ratio of the exclusion region to the specific region, which is calculated by the ratio calculation unit 111. If the ratio is used, the operation region setting unit 112 sets, as the calculation region, a region obtained by reducing the specific region based on the ratio in the image representing the characteristic of the material.

Using a high-energy radiation image X_{H} and a low-energy radiation image X_{L}, the physical amount calculation unit 113 calculates the surface density of the region (a soft tissue (fat) or a bone) generated by the material characteristic calculation unit 110. Since a value obtained by multiplying a thickness by a volume density is a surface density, the thickness and the surface density (to be also simply referred to as “density” hereinafter) substantially have equivalent meaning.

The physical amount calculation unit 113 calculates a material (soft tissue or bone) density using, of the radiation images corresponding to the plurality of energies, a radiation image (the low-energy radiation image X_{L} or the high-energy radiation image X_{H}) corresponding to one energy and the mass attenuation coefficient of the material (soft tissue or bone) corresponding to one energy. The physical amount calculation unit 113 calculates the physical amount representing the characteristic of the material forming the object 103 in the calculation region set by the operation region setting unit 112. If the specific region is a bone region forming the object 103, the physical amount calculation unit 113 calculates a bone density as the physical amount representing the characteristic of the material.

The reporting output unit 114 (output processing unit) outputs the physical amount (for example, the bone density) representing the characteristic of the material, which is calculated by the physical amount calculation unit 113. The calculation result of the physical amount (bone density) representing the characteristic of the material, which is output from the reporting output unit 114, is input to the control unit 105, and the control unit 105 causes the monitor 106 to display a report concerning the calculation result of the physical amount (bone density) representing the characteristic of the material.

(Processing Procedure in Image Processing Unit 109)

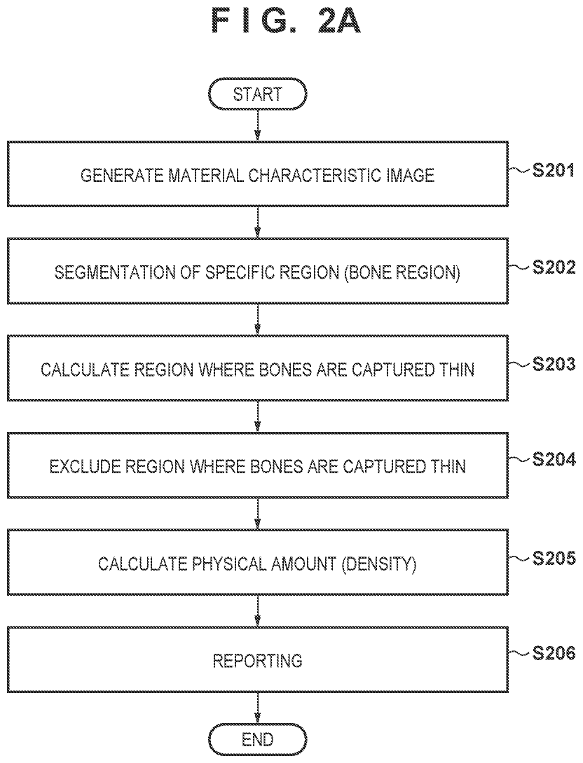

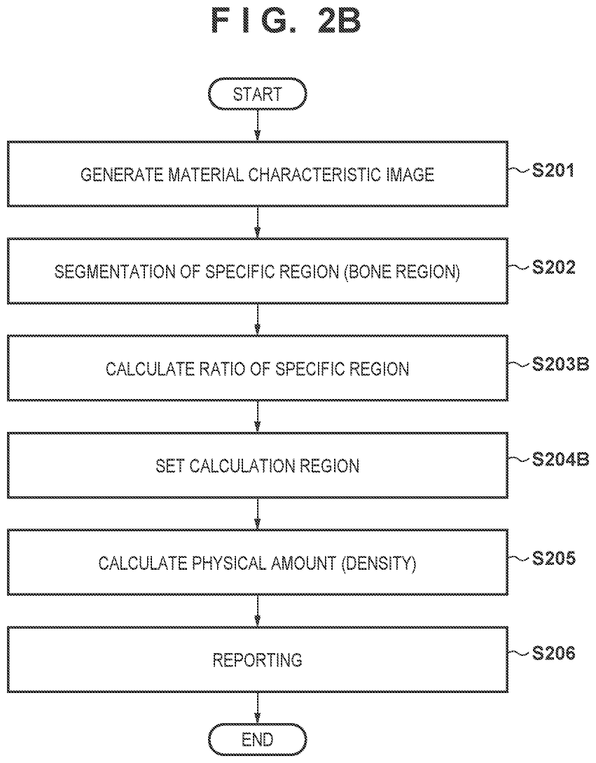

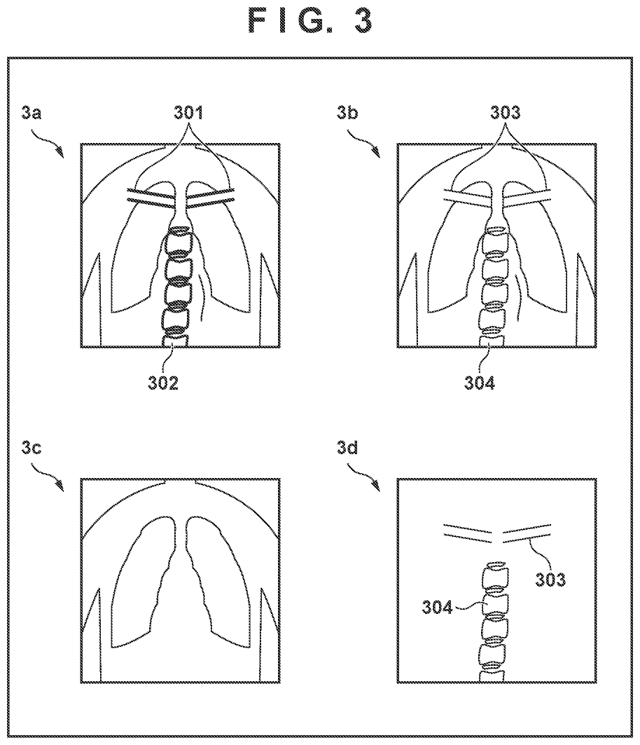

Processing in the image processing unit 109 according to the first embodiment will be described next in detail with reference to the flowchart shown in FIG. 2A. The control unit 105 stores, in the storage unit 108, a radiation image captured by the FPD 102 and transfers the radiation image to the image processing unit 109. 3a of FIG. 3 is a view showing a high-energy radiation image, and 3b of FIG. 3 is a view showing a low-energy radiation image. Also, 3c of FIG. 3 is a view showing the material decomposition image of soft tissues, and 3d of FIG. 3 is a view showing the material decomposition image of bones.

In the following processing, a fat image and a bone image will be described as material decomposition images, that is, images obtained by decomposing the object 103 into two or more specific materials. However, this embodiment is not limited to this example, and the processing can be similarly applied even if the object is decomposed to other materials, or the object is decomposed to an effective atomic number and a surface density.

(S201: Generation of Material Characteristic Image)

First, in step S201, the material characteristic calculation unit 110 generates material decomposition images that are material characteristic images. More specifically, based on equations (1) and (2) below, the material characteristic calculation unit 110 generates material decomposition images from the high-energy radiation image X_{H} shown in 3a of FIG. 3 and the low-energy radiation image X_{L} shown in 3b of FIG. 3, which are captured by the FPD 102. Bone portions (collar bones 303 and spinal bones 304) in the low-energy radiation image X_{L} shown in 3b of FIG. 3 are displayed with clear contrast as compared to bone portions (collar bones 301 and spinal bones 302) in the high-energy radiation image X_{H} shown in 3a of FIG. 3.

−ln X_{L}=μ_{LA}d_{A}+μ_{LB}d_{B} (1)

−ln X_{H}=μ_{HA}d_{A}+μ_{HB}d_{B} (2)

where μ is a ray attenuation coefficient, d is the thickness of a material, subscripts H and L represent high energy and low energy, respectively, and subscripts A and B represent materials to be decomposed, respectively (for example, A represents fat as a soft tissue, and B represents bones). μ_{HA} is the ray attenuation coefficient of soft tissues (fat) at high energy, and μ_{HB} is the ray attenuation coefficient of bones at high energy. Also, μ_{LA} is the ray attenuation coefficient of soft tissues (fat) at low energy, and μ_{LB} is the ray attenuation coefficient of bones at low energy.

Here, as examples of materials to be decomposed, soft tissues (fat) and bones are used as examples of materials. However, the materials are not particularly limited, and arbitrary materials can be used. The material characteristic calculation unit 110 performs arithmetic processing of solving the simultaneous equations of equations (1) and (2), thereby obtaining equations (3) below. Material decomposition images decomposed to the materials can thus be obtained. 3c of FIG. 3 is a view showing a material decomposition image obtained based on a thickness d_{A} of soft tissues (fat) in equation (3), and 3d of FIG. 3 is a view showing a material decomposition image obtained based on a thickness d_{B} of bones in equation (3).

$${d}_{A} = \frac{1}{{μ}_{LA} {μ}_{HB} - {μ}_{LB} {μ}_{HA}} ( {μ}_{LB} ln {X}_{H} - {μ}_{HB} ln {X}_{L} ) \\ {d}_{B} = \frac{1}{{μ}_{LB} {μ}_{HA} - {μ}_{HB} {μ}_{LA}} ( {μ}_{LA} ln {X}_{H} - {μ}_{HA} ln {X}_{L} ) \qquad ( 3 )$$

(S202: Segmentation of Specific Region (Bone Region))

In step S202, the material characteristic calculation unit 110 calculates a specific region from the material decomposition images generated in step S201. In this step, the material characteristic calculation unit 110 specifies a specific region based on radiation images output from the FPD 102 (radiation detector) by a plurality of times of radiation irradiation using different tube voltages. The material characteristic calculation unit 110 calculates, from the bone image that is a material decomposition image, a bone region as a specific region made of a specific material forming the object 103. The bone image d_{B} as shown in 3d of FIG. 3 does not include soft tissues as shown in 3c of FIG. 3. For this reason, a bone region in the bone image d_{B} can be specified by performing, for example, histogram analysis or threshold processing. As the threshold processing, for example, binarization can be used. The bone region can also be specified using region extension, edge detection, or graph cut, which are known techniques. If many radiation images with the object captured can be obtained, the specific region (bone region) may be specified using a region extraction method (segmentation processing) by machine learning (deep learning) using the radiation images as supervisory data. There may be a function of allowing a technician to correct the automatically set bone region using known image processing software.

In this embodiment, to facilitate specifying of a region, the bone image d_{B} is used. However, the present invention is not limited to this, and a bone region and a region including only soft tissues may be specified from the high-energy radiation image X_{H} and the low-energy radiation image X_{L}, respectively.

(S203: Calculation of Region (Exclusion Target Region) Where Bones Are Captured Thin)

In step S203, the operation region setting unit 112 calculates a region where bones are captured thin by incidence (oblique incidence) of X-rays from an oblique direction as an exclusion target region from the bone region calculated in step S202. Here, the region where bones are captured thin is a region where the pixel values are lower than a predetermined threshold in the bone image d_{B}.

The operation region setting unit 112 calculates, as the exclusion target region, a range having pixel values lower than a predetermined threshold in the specific region. The operation region setting unit 112 specifies, as the region (exclusion target region) where bones are captured thin, a region having pixel values lower than a predetermined threshold in the bone region of the bone image d_{B} of the object irradiated with radiation.

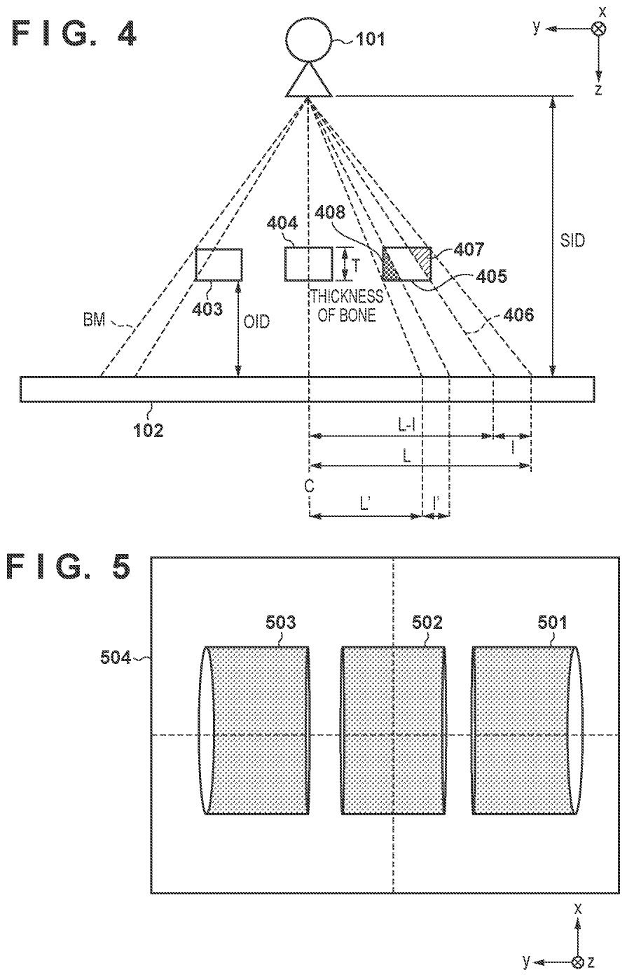

FIG. 4 is a view for explaining a geometric arrangement representing the relative positional relationship of the radiation tube 101, the object, and the FPD 102 (radiation detector). A Z-axis is set vertically downward from the radiation tube 101, a y-axis is set in the longitudinal direction (lateral direction) of the FPD 102, and an x-axis is set in a direction perpendicular to the sheet surface. X-rays generated by the radiation generating apparatus 104 have a spread from the radiation tube 101 toward the object 103 (BM in FIG. 4), and some components of the radiation pass through the object 103 (lumbar vertebrae 403 to 405) and reach the FPD 102.

As shown in FIG. 4, based on the geometric arrangement (relative positional relationship) of the radiation tube 101, the FPD 102 (radiation detector), and the object 103, the operation region setting unit 112 can calculate ranges (exclusion target regions I and I′) having pixel values lower than a predetermined threshold using equations (4) and (5).

I=T/(SID−OID)×L (4)

I′=T/(SID−OID−T)×L′ (5)

Referring to FIG. 4, the exclusion target region I is a region where bones are captured thin in the bone image d_{B} by oblique incidence of the radiation (the radiation that has entered the portion of a region 407 in FIG. 4). A parameter L representing the length (distance) in the lateral direction (y-axis direction) indicates a distance corresponding to that from a center C of the FPD 102 (radiation detector) to the outer frame (side end portion) of the bone region calculated in step S202. The parameter L can be calculated from the bone region calculated in step S202, and is obtained using equation (4). Also, the exclusion target region I′ is a region where bones are captured thin in the bone image d_{B} by oblique incidence of the radiation (the radiation that has entered the portion of a region 408 in FIG. 4), which is a region when the exclusion region I exists in the centrifugal direction. A parameter L′ representing the length (distance) in the lateral direction (y-axis direction) indicates a distance corresponding to that from the center C of the FPD 102 (radiation detector) to the outer frame (side end portion) of the bone region calculated in step S202. The parameter L′ can be calculated from the bone region calculated in step S202, and is obtained using equation (5). In this embodiment, only one direction has been described. The actual operation is needed in both the X and Y directions, and the operation is performed for the whole bone region calculated in step S202 or a thinned outer peripheral portion.

SID (Source to Image Distance) represents the distance between the radiation tube 101 and the FPD 102 (radiation detector), and OID (Object to Image Distance) represents the distance from the object 103 (in the example shown in FIG. 4, the bone region (lumbar vertebrae 403 to 405)) to the FPD 102 (radiation detector). SID and OID can be set as fixed values, and a user or a serviceman can also input SID and OID using the operation unit 107. The operation region setting unit 112 obtain the geometric arrangement (relative positional relationship) based on the distance (SID) between the radiation tube and the FPD 102 (radiation detector) and the distance (OID) between the object 103 and the FPD 102 (radiation detector).

In addition, as a bone thickness T, a statistically average bone thickness can be preset. The bone thickness can also be calculated from the generated material decomposition image (bone image).

(S204: Exclusion of Region Where Bones Are Captured Thin (Setting of Calculation Region))

In step S204, the operation region setting unit 112 excludes the range (exclusion target region I) where bones are captured thin, which is calculated in step S203, from the specific region (bone region L) calculated in step S202, and sets the reduced specific region (bone region (L−I)) as a calculation region in the image representing the characteristic of the material.

At this time, the operation region setting unit 112 deletes, from position information for defining the specific region (bone region), the position information of the range (exclusion target region I) where bones are captured thin, which is calculated in step S203, to update the position information of the specific region (bone region), and sets the reduced specific region (bone region (L−I)) as a calculation region in the image (material decomposition image) representing the characteristic of the material. In addition, the operation region setting unit 112 performs contraction processing by morphology conversion for the specific region (bone region) calculated in step S202 to exclude the region (exclusion target region) where bones are captured thin, which is calculated in step S203, from the specific region (bone region) calculated in step S202 and sets the reduced specific region (bone region (L−I)) as a calculation region in the image representing the characteristic of the material. The radiation image of the reduced specific region (bone region (L−I): calculation region) corresponds to an image captured by radiation 406 shown in FIG. 4.

(S205: Calculation of Physical Amount (Density))

In step S205, the physical amount calculation unit 113 calculates a physical amount (density) representing the characteristic of the material forming the object 103 in the calculation region set in step S204. The physical amount calculation unit 113 calculates the physical amount (density) representing the characteristic of a material (for example, bones) forming the object 103 in the calculation region (bone region (L−I)) using the radiation image (low-energy radiation image X_{L}(x,y) or the high-energy radiation image X_{H}(x,y)) corresponding to one energy in the radiation images of the plurality of energies and the mass attenuation coefficient of the material corresponding to one energy.

By modifying equation (1), the physical amount calculation unit 113 can calculate the physical amount (bone density) representing the characteristic of the material in the set calculation region (bone region (L−I)) based on the calculation of low-energy radiation image (−ln X_{L}(x,y))/(mass attenuation coefficient of bones at low energy). In each material decomposition image generated in step S201, since it is known that the region is made of only a specific material (for example, bones or soft tissue), simple calculation as described above can hold.

Similarly, by modifying equation (2), the physical amount calculation unit 113 can calculate the physical amount (bone density) representing the characteristic of the material in the set calculation region (bone region (L−I)) based on the calculation of high-energy radiation image X_{H}(x,y)/(mass attenuation coefficient of bones at high energy). Note that the processing of the physical amount calculation unit 113 can be applied to calculate the density value not only in the bone region but also in the soft tissue region.

(S206: Reporting: Output Processing)

In step S206, the reporting output unit 114 (output processing unit) outputs the bone density value calculated by the physical amount calculation unit 113 in step S205. The calculation result of the bone density value output from the reporting output unit 114 (output processing unit) is input to the control unit 105, and the control unit 105 causes the monitor 106 to display a report concerning the calculation result of the control unit value. The series of processes in the image processing unit 109 thus ends.

(Modification of Processing Procedure in Image Processing Unit 109)

A modification of the processing procedure in the image processing unit 109 according to the first embodiment will be described next. FIG. 2B is a flowchart showing a modification of the processing procedure by the image processing unit 109 according to the first embodiment. The processing procedure shown in FIG. 2B is different from the processing procedure shown in FIG. 2A in that in step S203, the ratio calculation unit 111 calculates the ratio of the exclusion region to the specific region in the image (material characteristic image) representing the characteristic of the material, and in step S204, the operation region setting unit 112 sets the calculation region in the image representing the characteristic of the material based on the ratio.

(S203B: Calculation of Ratio of Specific Region)

In step S203B of FIG. 2B, the ratio calculation unit 111 calculates the ratio of the exclusion region to the specific region in the image (material characteristic image) representing the characteristic of the material based on the geometric arrangement representing the relative positional relationship of the radiation tube 101, the FPD 102 (radiation detector), and the object 103. In this step, the ratio calculation unit 111 obtains a geometric arrangement (relative positional relationship) as shown in FIG. 4 based on the distance (SID) between the radiation tube 101 and the FPD 102 (radiation detector) and the distance (OID) between the object 103 and the FPD 102 (radiation detector). Referring to FIG. 4, the exclusion target region I can be obtained based on equation (4) based on the geometric arrangement (relative positional relationship), and the parameter L representing the length (distance) in the lateral direction (y-axis direction) can be calculated from the bone region calculated in step S202. The ratio calculation unit 111 obtains the result of dividing the parameter L obtained from the bone region by (L−I) as the ratio (EG=L/(L−I)) of the exclusion region to the specific region.

(Step S204B: Setting of Calculation Region to Calculate Physical Amount)

In step S204B of FIG. 2B, based on the ratio of the exclusion region to the specific region, which is calculated in step S203B, the operation region setting unit 112 sets the calculation region to calculate the physical amount representing the characteristic of the material forming the object 103 in the image (material decomposition image) representing the characteristic of the material. In this step, the operation region setting unit 112 sets, as the calculation region, a region (bone region (L−I)) obtained by reducing the specific region (bone region L) based on the ratio EG of the exclusion region to the specific region as the calculation region in the image representing the characteristic of the material.

The same processing as in FIG. 2A is performed from step S205. In the calculation region set in step S204B, the physical amount calculation unit 113 calculates the physical amount (density) representing the characteristic of the material forming the object 103.

Note that in the description of FIG. 4, lumbar spine imaging of the object 103 has been described as an example. As for the part to measure the bone density, measurement is recommended to be done in a thigh bone in addition to the lumbar spine. In this embodiment, the processing can be applied to the thigh bone as well in accordance with the same procedure as that of lumbar spine imaging, and the processing can be applied to any part in the object 103.

In a situation in which the geometric arrangement representing the relative positional relationship of the radiation tube 101, the object, the FPD 102, and the FPD (radiation detector) is known, it is possible, by applying the processing according to the first embodiment, exclude the exclusion target region I from the specific region (for example, the bone region) and set the reduced specific region (bone region (L−I)) as the calculation region in the image representing the characteristic of the material (FIG. 2A). It is also possible to calculate the enlargement ratio EG based on the geometric arrangement (relative positional relationship) and, based on the enlargement ratio EG, set the reduced specific region (bone region (L−I)) as the calculation region in the image representing the characteristic of the material (FIG. 2B).

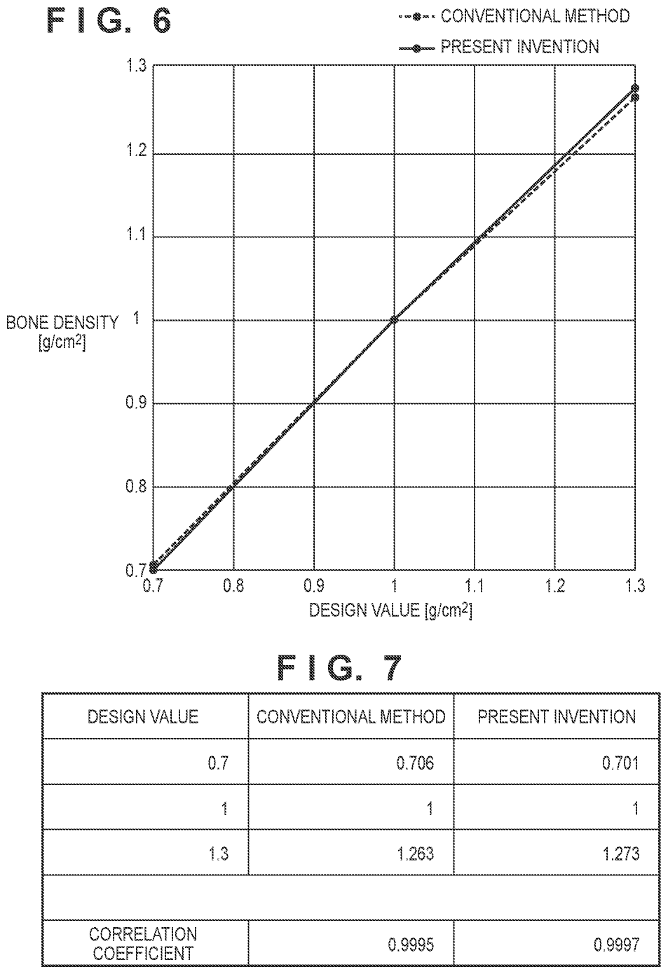

The effects of the first embodiment will be described next with reference to FIGS. 5, 6, and 7. FIG. 5 is a view showing an X-ray image obtained by capturing a lumbar spine phantom, and FIGS. 6 and 7 are views showing the effects according to the first embodiment.

The X-ray image shown in FIG. 5 is an X-ray image obtained by capturing a lumbar spine phantom that imitates a human body with a body thickness of 15 cm. A frame 504 indicates the outer frame of the effective imaging region of the FPD 102. In the lumbar spine phantom, a lumbar spine (L2) 501 with a bone density of 0.7 g/cm², a lumbar spine (L3) 502 with a bone density of 1.0 g/cm², and a lumbar spine (L4) 503 with a bone density of 1.3 g/cm² are buried in the lumbar spine phantom. The lumbar spine phantom is captured using high-energy radiation and low-energy radiation, and a graph obtained by calculating the bone densities of the lumbar vertebrae is shown in FIG. 6.

In the graph shown in FIG. 6, the ordinate represents the calculated bone density value, and the abscissa represents the design value (bone density value) of the phantom. In FIG. 6, bone density values calculated by processing as described in PTL 1 are plotted as a conventional method by a solid line, and bone density values calculated by the processing according to the first embodiment are plotted as the present invention by a broken line.

FIG. 7 is a view that compares the numerical values in the graph of FIG. 6. As shown in FIG. 6, values close to the design values can be obtained using not the conventional method but the processing according to the first embodiment. Also, if the correlation coefficients between the design values and the bone density calculation values are compared by statistical processing, the correlation coefficient in the conventional method is 0.9995, and the correlation coefficient in the present invention is 0.9997. The correlation coefficient of the bone density value calculated by the processing according to the first embodiment of the present invention is improved as compared to the correlation coefficient of the bone density value calculated by the conventional method. According to the processing of the first embodiment of the present invention, the change of the bone density of phantom can more correctly be calculated.

As described above, according to the first embodiment, even in enlargement imaging using a fan beam, a cone beam, or the like, it is possible to more correctly calculate a physical amount representing the characteristic of a material forming an object. For example, it is possible to more correctly calculate the bone density of bones as the material forming the object.

### Second Embodiment

In the first embodiment, an example in which distance information (geometric information) such as SID or OID in the geometric arrangement (relative positional relationship) is given to the information processing apparatus 120 has been described. However, there may be a situation in which geometric information such as SID or OID cannot be obtained or a case in which geometric information such as SID or OID cannot be input from the viewpoint of reducing the burden on the user.

In the second embodiment, a configuration will be described, in which a region (exclusion target region) where bones are captured thin is specified using image processing and excluded from a specific region (for example, a bone region), and a reduced specific region (bone region (L−I)) is set as a calculation region in an image representing the characteristic of a material. Concerning the second embodiment, points different from the first embodiment will be described in detail. The basic configuration of a radiation imaging system is the same as the radiation imaging system 100 (FIG. 1) described in the first embodiment. In the following explanation, a description of the same parts as in the first embodiment will be omitted, and processing specific to the second embodiment will be described.

In the second embodiment, the processes of steps S201 and S202 and the processes of steps S204 to S206 in FIG. 2A are the same as in the first embodiment. In the process of step S203, a region (exclusion target region) where bones are captured thin is specified based on a result of image processing (image analysis) without using geometric information, unlike the processing according to the first embodiment.

(S203: Calculation of Region (Exclusion Target Region) Where Bones Are Captured Thin)

In step S203, based on the result of image processing, an operation region setting unit 112 specifies, from the bone region calculated in step S202, a region (exclusion target region) where bones are captured thin by incidence (oblique incidence) of X-rays from an oblique direction. Based on the result of image analysis of the image representing the characteristic of the material, the operation region setting unit 112 calculates the range (exclusion target region I) where bones are captured thin. The operation region setting unit 112 obtains, by image analysis, a region representing a predetermined pixel value and a region where the predetermined pixel value changes to cause inclination in the image representing the characteristic of the material, and calculates, based on the position information of the region where the pixel value has changed, the range (exclusion target region I) where bones are captured thin.

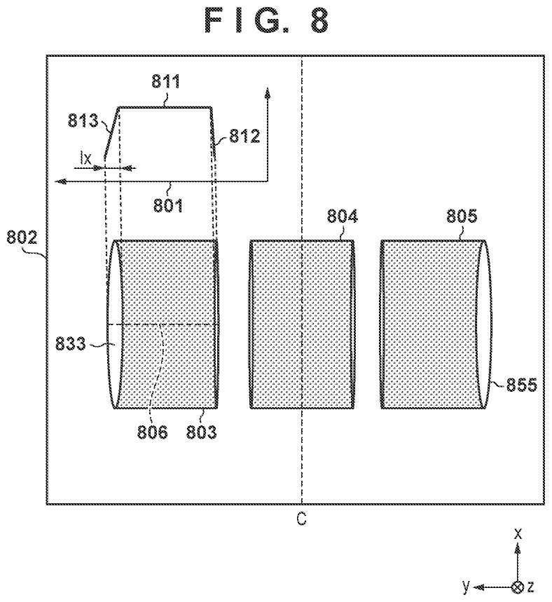

FIG. 8 is a view for explaining a processing method according to the second embodiment. Referring to FIG. 8, a frame 802 indicates the outer frame of the effective imaging region of an FPD 102. As shown in FIG. 8, in lumbar spine imaging, as the distance from a center C of the FPD 102 (radiation detector) in the longitudinal direction (lateral direction: y-axis direction) of the FPD 102 becomes long, the region (exclusion target region) where bones are captured thin may be generated. Side end portions (833 and 855) of lumbar vertebrae 803 and 805 can be regions where an exclusion target region is readily generated as compared to a lumbar spine 804 located at the center.

In the effective imaging region (x-y plane), the operation region setting unit 112 obtains a profile representing the two-dimensional distribution of the pixel values of a bone portion in a bone image. Referring to FIG. 8, a profile 801 represents the distribution of the pixel values of the lumbar spine 803 along a broken line 806 (a y-axis direction that is a body axis direction). The profile 801 has a profile output 811 where a predetermined pixel value is obtained, and profile outputs 812 and 813 where the predetermined pixel value changes to cause inclination.

In a portion where bones are captured thin, inclination always occurs in the pixel value output. Using this feature, in the specific region (bone region) calculated in step S202, the operation region setting unit 112 obtains a profile in the body axis direction (j-axis direction) and specifies a portion where the inclination is not constant. For example, in the profile 801, inclination occurs in the profile outputs 812 and 813. Based on the position information of the pixels in the specific region (bone region), the operation region setting unit 112 specifies a region Ix (exclusion target region) where bones are captured thin, based on the profile output 813 located on a side end portion side in the specific region (bone region). The region Ix specified based on image analysis by the operation region setting unit 112 is a region corresponding to the region I in FIG. 4.

When executing image analysis, the operation region setting unit 112 may perform smoothing of a profile so that a profile extraction error does not occur. Alternatively, for a plurality of lumbar vertebrae, the operation region setting unit 112 may obtain a profile in a direction crossing a direction obtained by collecting the body axis directions (y-axis directions).

The operation region setting unit 112 applies the image analysis to all bone regions, thereby specifying, based on the result of image analysis, the region (exclusion target region) where bones are captured thin by enlargement imaging in the specific region (bone region) calculated in step S202.

Also, the operation region setting unit 112 may specify the exclusion target region by threshold processing in accordance with the pixel values in the bone region, or the bone thickness or bone density. As for the threshold, the Otsu's method may be used in the bone region. When using the bone thickness or bone density, a threshold equal to or less than ⅓ of a standard value may be provided. The exclusion target region exists only in a marginal portion because of the feature of enlargement imaging. For this reason, when processing by morphology conversion is performed, the inside of the bone region can be prevented from being erroneously excluded.

According to the processing of the second embodiment, it is possible to specify the region (exclusion target region) where bones are captured thin based on a result of image processing (image analysis) without using geometric information. When the operation result of the operation region setting unit 112 in the processing according to the second embodiment is applied to processing from step S204 in FIG. 2A, the same effects as in the first embodiment can be obtained.

According to the second embodiment, even in enlargement imaging using a fan beam, a cone beam, or the like, it is possible to more correctly calculate a physical amount representing the characteristic of a material forming an object. For example, it is possible to more correctly calculate the bone density of bones as the material forming the object.

### Third Embodiment

In the process of step S203 described in the first embodiment, an example in which geometric information is used when calculating the region (exclusion target region) where bones are captured thin has been described. Also, in the second embodiment, an example of processing of specifying the exclusion target region based on the result of image processing (image analysis) without using geometric information has been described.

In the processing according to the second embodiment, however, the analysis accuracy may be affected by the image quality of a bone image or the shape of a bone. For this reason, the region where bones are actually captured thin as an image may not match the range of the exclusion target region I obtained by equation (4).

In such a case, an operation region setting unit 112 can specify the exclusion target region by combining the result of image processing (image analysis) and the geometric information. In this embodiment, the operation region setting unit 112 specifies, by image processing (image analysis), the region (exclusion target region) where bones are captured thin. At this time, the operation region setting unit 112 can use a result obtained from the geometric information as a reference value used to determine whether a change has occurred in the image analysis result.

If the result of image analysis does not match the result obtained from a geometric arrangement (relative positional relationship), the operation region setting unit 112 calculates, based on the position information of pixels obtained from the geometric arrangement (relative positional relationship), the range (exclusion target region I) where bones are captured thin.

For example, the operation region setting unit 112 can specify, using a result obtained from geometric information, a position where a profile 801 representing a predetermined pixel value has changed. If a plurality of candidates of the position where the profile 801 has changed are obtained based on the result of image analysis, using position information that is most suitable to the result obtained from the geometric information, the operation region setting unit 112 specifies profile outputs 812 and 813 where the pixel value changes to cause inclination.

According to this embodiment, when the result of image processing (image analysis) and the geometric information are combined, the region (exclusion target region) where bones are captured thin can be more correctly specified. When the operation result of the operation region setting unit 112 in the processing according to the third embodiment is applied to processing from step S204 in FIG. 2A, the same effects as in the first and second embodiments can be obtained.

According to the third embodiment, even in enlargement imaging using a fan beam, a cone beam, or the like, it is possible to more correctly calculate a physical amount representing the characteristic of a material forming an object. For example, it is possible to more correctly calculate the bone density of bones as the material forming the object.

According to the first embodiment, the second embodiment, and the third embodiment, it is possible to more correctly calculate a physical amount representing the characteristic of a material forming an object. For example, it is possible to more correctly calculate the bone density of a bone as a material forming an object.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus for processing a radiation image, comprising
a calculation unit configured to calculate, in a calculation region, a physical amount representing a characteristic of a material, the calculation region being obtained using (a) a specific region regarding a specific material in an image representing the characteristic of the material and (b) a relative positional relationship of a radiation tube, a radiation detector, and an object, wherein the image representing the characteristic of the material is obtained using information about a plurality of radiation energies.

2. The image processing apparatus according to claim 1, wherein the relative positional relationship is obtained based on a distance between the radiation tube and the radiation detector and a distance between the object and the radiation detector.

3. The image processing apparatus according to claim 1, wherein the calculation region is obtained using a range having a pixel value lower than a threshold in the specific region and the relative positional relationship.

4. An image processing apparatus for processing a radiation image, comprising
a calculation unit configured to calculate, in a calculation region obtained using a range having a pixel value lower than a threshold in a specific region concerning a specific material in an image representing a characteristic of a material, which is obtained using information about a plurality of radiation energies, a physical amount representing the characteristic of the material.

5. The image processing apparatus according to claim 4, wherein the calculation region is obtained using the range and a relative positional relationship of a radiation tube, a radiation detector, and an object.

6. The image processing apparatus according to claim 5, wherein the relative positional relationship is obtained based on a distance between the radiation tube and the radiation detector and a distance between the object and the radiation detector.

7. The image processing apparatus according to claim 3, wherein the calculation unit calculates the range based on a result of image analysis of the image representing the characteristic of the material.

8. The image processing apparatus according to claim 3, wherein the calculation unit obtains a region representing a predetermined pixel value and a region where the predetermined pixel value changes to cause inclination in the image representing the characteristic of the material by image analysis of the image representing the characteristic of the material, and calculates the range based on position information of the region where the pixel value has changed.

9. The image processing apparatus according to claim 3, wherein if a result of image analysis of the image representing the characteristic of the material and a result obtained from the relative positional relationship do not match, the calculation unit calculates the range based on position information of a pixel obtained from the relative positional relationship.

10. The image processing apparatus according to claim 3, wherein the calculation region is obtained by reducing, using the relative positional relationship, a region obtained by excluding the range from the specific region.

11. The image processing apparatus according to claim 1, wherein the specific region is specified based on radiation images output from the radiation detector by a plurality of times of radiation irradiation using different tube voltages.

12. The image processing apparatus according to claim 1, wherein the specific region is specified using a region extraction method by machine learning for radiation images of the plurality of energies.

13. The image processing apparatus according to claim 1, wherein the calculation unit calculates a density as the physical amount representing the characteristic of the material using, of radiation images corresponding to the plurality of energies, a radiation image corresponding to one energy and a mass attenuation coefficient of the material corresponding to the one energy.

14. The image processing apparatus according to claim 13, wherein
the specific region is a bone region forming the object, and
the calculation unit calculates a bone density as the physical amount represent the characteristic of the material.

15. A radiation imaging apparatus comprising:
an image processing apparatus defined in claim 1; and
a radiation detector
such that the image processing apparatus and the radiation detector can communicate.

16. An image processing method for processing a radiation image, comprising
calculating, in a calculation region, a physical amount representing a characteristic of a material, the calculation region being obtained using (a) a specific region regarding a specific material in an image representing the characteristic of the material and (b) a relative positional relationship of a radiation tube, a radiation detector, and an object, wherein the image representing the characteristic of the material is obtained using information about a plurality of radiation energies.

17. An image processing method for processing a radiation image, comprising
calculating, in a calculation region obtained using a range having a pixel value lower than a threshold in a specific region concerning a specific material in an image representing a characteristic of a material, which is obtained using information about a plurality of radiation energies, a physical amount representing the characteristic of the material.

18. A non-transitory computer readable storage medium storing a program for causing a computer to execute steps in the method according to claim 16.

19. A non-transitory computer readable storage medium storing a program for causing a computer to execute steps in the method according to claim 17.
